(19) United States
(12) Patent Application Publication
(10) Pub. No.: US 2024/0365720 A1
(43) Pub. Date: Nov. 7, 2024

(54) COMPOSITIONS AND METHODS FOR MITIGATING HLB

(71) Applicants: THE STATE OF ISRAEL, MINISTRY OF AGRICULTURE & RURAL DEVELOPMENT, AGRICULTURAL, Rishon-LeZion (IL); University of Florida Research Foundation, Incorporated, Gainesville, FL (US); United States of America, as represented by the Secretary of Agriculture, Washington, DC (US)

(72) Inventors: Eran RAVEH, Midreshet Ben-Gurion (IL); Ute ALBRECHT, Sebastian, FL (US); Kim D. BOWMAN, Port St. Lucie, FL (US); Zipora TIETEL, Givot Bar (IL)

(73) Assignees: THE STATE OF ISRAEL, MINISTRY OF AGRICULTURE & RURAL DEVELOPMENT, AGRICULTURAL, Rishon-LeZion (IL); University of Florida Research Foundation, Incorporated, Gainesville, FL (US); United States of America, as represented by the Secretary of Agriculture, Washington, DC (US)

(21) Appl. No.: 18/689,097

(22) PCT Filed: Sep. 5, 2022
(86) PCT No.: PCT/IL2022/050965

§ 371 Date: Mar. 5, 2024

Related U.S. Application Data
(60) Provisional application No. 63/325,179, filed on Mar. 30, 2022.
(60) Provisional application No. 63/240,943, filed on Sep. 5, 2021.

(51) Int. Cl. A01G 2/30; A01H 5/08; A01H 6/78

(52) U.S. Cl. CPC A01G 2/30; A01H 5/08; A01H 6/785

(57) ABSTRACT

A composition comprising an HLB-susceptible or tolerant citrus rootstock grafted with an HLB-tolerant or resistant citrus interstock is provided. Also provided are plants comprising same and uses thereof in citrus growth.

### RELATED APPLICATION

This application claims the benefit of priority of U.S. Provisional Patent Application No. 63/240,943 filed on 5 Sep. 2021 and U.S. Provisional Patent Application No. 63/325,179 filed 30 Mar. 2022, the contents of which are incorporated herein by reference in their entirety.

### FIELD AND BACKGROUND OF THE INVENTION The present invention, in some embodiments thereof, relates to compositions and methods for mitigating huanglongbing (HLB).

Citrus is one of the top specialty crops grown in the United States and Israel, ranked third in terms of bearing area and accounts for 16% of the total value of U.S. fruit production, and 20% in Israel (FAO, 2020; Israel Farmer Federation, 2015). Citrus greening, also called huanglongbing or HLB, is a bacterial disease of the phloem spread by an invasive insect vector, the Asian citrus psyllid (Garnier et al., 1984; Hall et al., 2013). In the U.S. and most other citrus producing countries the pathogen associated with HLB is Candidatus Liberibacter asiaticus (CLas). Thus far the pathogen has not been successfully grown in pure culture to fulfill Koch's postulate and complicating experimentation (Merfa et al., 2019).

The initial infection of trees with CLas is uneven and trees often do not exhibit disease symptoms until many months or years after infection. Affected trees typically exhibit nutrient deficiency-like symptoms, leaf yellowing and canopy thinning, followed by shoot dieback and trec decline (Wang 2019; Gottwald et al, 2007; Bove, 2006). In addition to tree decline, fruit on infected trees remain small with poor coloration and poor internal quality (Liao and Burns, 2012; Baldwin et al., 2010). In Florida, where HLB is ubiquitous, one of the most devastating disease effects is preharvest fruit drop, which leads to tremendous fruit losses during the months before harvest, exacerbating losses from the reduced fruit quality.

HLB has upended the citrus-growing industry in Florida, put other American citrus producing regions, like California and Texas, on high alert, and decimated citrus industries worldwide (Gottwald et al., 2007; Graham et al., 2020). HLB is endemic in Florida (Graham et al., 2020) and since its discovery in 2005, Florida citrus production has been reduced by more than 70%, causing substantial economic losses (Singerman et al., 2018; Rosson, 2020). Methods of mitigating the disease impacts include vector management, nutritional therapies, and other horticultural practices (Stansly et al., 2014) but none of these have been effective.

Most citrus scion cultivars are highly susceptible to HLB (McClean and Schwarz, 1970; Shokrollah et al., 2009), but tolerance was identified in some hybrids of citrus and trifoliate orange (Poncirus trifoliata) that are commonly used as rootstocks (Albrecht and Bowman, 2011, 2012; Folimonova et al., 2009; Ramadugu et al., 2016). Although a tolerant rootstock does not prevent the grafted scion from becoming infected, using HLB-tolerant rootstocks can help affected trees to cope better with the detrimental effects of the disease and increase the lifespan and therefore profitability of a citrus orchard. Hence, the demand for new and superior rootstocks has dramatically increased since the arrival of HLB in Florida, and field trials have demonstrated that the use of specific rootstocks can increase the productivity of commercially grown citrus trees in an HLB-endemic environment (Kunwar et al., 2021; Bowman et al., 2016a, 2016b; Boava et al. 2015; Albrecht et al., 2014).

In addition to trifoliate orange, which is considered HLB tolerant, finger limes (Microcitrus australasica) and several other species in that genus have demonstrated field tolerance to HLB (Dutt et al., 2017a, 2017b; Ramadugu et al., 2016). Finger limes were even suggested as a new crop for citrus growers in Florida (Dutt et al., 2017b). One of the reasons for the higher tolerance or resistance observed for some citrus relatives is a lower preference of the insect vector for feeding and reproduction (Hernandez-Suarez et al, 2021; Luo et al 2015). In addition to the Asian citrus psyllids' low preference for finger limes, the trees were shown to have a low infection rate, low titers of the HLB bacterium, and few foliar symptoms of the disease after six years of field evaluation (Ramadugu et al., 2016). Long-term field evaluation also demonstrated a high tolerance of Microcitrus inodora (Large-leaf Australian wild lime) (Ramadugu et al., 2016). In 2018, ‘Minnie’ finger lime, a new cultivar resulting from a cross of M. australasica and M. inodora, was released by USDA for commercial use based on strong growth and fruit production when grown under HLB-endemic conditions in Florida (Bowman et al., 2019).

Recently, a peptide was suggested to be associated with the tolerance or resistance of species in the genus Microcitrus (Huang et al., 2021). The peptide was demonstrated to have a dual functionality comprising antimicrobial activity as well as priming activity; this combined activity results in suppression of the pathogen and the induction of plant immune/defense responses further suppressing the disease (Huang et al., 2021). The authors suggested that this peptide can be synthesized and applied to citrus trees by foliar spraying to reduce bacterial titers and reduce disease effects, but thus far this has not been demonstrated under HLB-endemic field conditions.

It has been previously suggested that volatile citrus compounds such as citronellol or different aldehydes in the phloem are associated with the reported tolerance of Microcitrus (Dutt, 2021). It has also been hypothesized that finger lime tolerance may be attributed to unfavorable visual clues caused by anthocyanins or by volatile emissions (Robinson, 2021). Specifically, the volatile compound β-caryophyllene was reported as a vector repellent (Alquezar et al, 2017) (although the authors used a β-caryophyllene from Arabidopsis rather than from Citrus). At the same time, a non-volatile citrus compound, hexaacetyl-chitohexaose, was shown to impart transient activation of plant defense, altering feeding behaviour of the insect vector (Shi et al, 2019). The phytochemical composition of the peel and pulp of four finger-limes has been published (Adhikari et al 2021), which may offer some valuable information regarding possible unique metabolites in finger limes responsible for tolerance. A study comparing the metabolic profiles of finger limes with HLB susceptible citrus cultivars identified several amino acids and sugars in the phloem sap that may be related to HLB tolerance (Killiny et al, 2018). A leaf transcriptomic study identified several genes for primary and secondary metabolites that were highly upregulated in finger limes but not in sweet orange trees, and which may be key to the higher tolerance of the former (Weber et al, 2019).

As with any crop protection material, spraying citrus trees with biochemicals such as antimicrobial or defense inducing compounds is expensive and will have to be conducted repeatedly to be effective. Furthermore, it has been demonstrated that foliar applications of antimicrobial chemicals are ineffective because they do not penetrate the thick citrus leaf cuticle to reach the phloem where the HLB pathogen resides (Killiny et al., 2020). Trunk injection as a delivery method for antimicrobials was shown to be effective in suppressing HLB (Da Graca et al. 1991 and references therein; Hu et al., 2018; Li et al., 2021), but can cause tree damage (Albrecht and Archer, 2021) and is impractical for large-scale use. An economically and environmentally more sustainable alternative to spraying or injecting citrus trees with the antimicrobial peptide is to use finger lime as a rootstock. However, finger limes are known for their low vigor and poor root adaptation to wet soils, and their feasibility as a rootstock is therefore low.

Background art includes U.S. patent application Ser. No. 20/190,364775.

### SUMMARY OF THE INVENTION

According to an aspect of the invention there is provided a composition comprising an HLB-susceptible or tolerant citrus rootstock grafted with an HLB-tolerant or resistant citrus interstock.

According to an aspect of the invention there is provided a composition comprising an HLB-susceptible or tolerant citrus rootstock grafted with an HLB-tolerant or resistant citrus interstock and an HLB-susceptible scion, wherein the HLB-tolerant citrus interstock is of a microcitrus or a hybrid thereof.

According to an aspect of the invention there is provided a method of producing an HLB tolerant citrus, the method comprising:
(a) grafting an HLB-tolerant or resistant citrus interstock on an HLB-susceptible or tolerant citrus rootstock grafted so as to obtain an HLB-susceptible or tolerant citrus rootstock grafted with the HLB-tolerant or resistant citrus interstock of a predetermined size, wherein the HLB-tolerant citrus interstock is of a microcitrus or a hybrid thereof; and
(b) grafting an HLB-susceptible scion on the HLB-susceptible or tolerant citrus rootstock grafted with the HLB-tolerant or resistant citrus interstock.

According to an aspect of the invention there is provided a method of producing an HLB tolerant or resistant citrus rootstock, the method comprising grafting an HLB-tolerant or resistant citrus interstock on an HLB-susceptible or tolerant citrus rootstock grafted so as to obtain an HLB-susceptible or tolerant citrus rootstock grafted with the HLB-tolerant or resistant citrus interstock of a predetermined size, wherein the HLB-tolerant citrus interstock is of a microcitrus or a hybrid thereof.

According to an aspect of the invention there is provided a composition comprising an HLB-susceptible or tolerant citrus rootstock grafted with a microcitrus which is HLB tolerant or resistant.

According to an aspect of the invention there is provided a method of producing fruits, the method comprising:
(a) growing a plant from the composition of claim 2; and
(b) harvesting fruits from the plant.

According to embodiments of the invention, the citrus is any cultivar from the genus Citrus, including but not limited to sweet orange (C. sinensis), lemon (C. limon), lime (C. latifolia) grapefruit (C. paradise), sour orange (C. aurantium), and mandarin (C. reticulata), trifoliate orange (Poncirus trifoliata) and any other citrus varieties and their hybrids.

According to embodiments of the invention, the interstock of the predetermined size is above 0.5 cm.

According to embodiments of the invention, the interstock of the predetermined size is above 0.6 cm.

According to embodiments of the invention, the HLB-tolerant citrus interstock is of a finger lime or hybrids thereof.

According to embodiments of the invention, the finger lime is Minnie finger lime or hybrids thereof.

According to embodiments of the invention, the scion is of Valencia sweet orange (C. sinensis).

According to embodiments of the invention, the rootstock is of Volkamer lemon [Citrus volkameriana] or US-942 (Citrus reticulata x Poncirus trifoliata).

According to embodiments of the invention, the rootstock is of US-942 (Citrus reticulata x Poncirus trifoliata).

According to embodiments of the invention, at least one of the rootstock, scion and interstock is diploid.

According to embodiments of the invention, the composition is a tree.

According to embodiments of the invention, the microcitrus is Minnie finger lime or hybrids thereof.

According to some embodiments of the invention, the method comprises:
(a) growing a plant from the composition as described herein; and
(b) harvesting fruits from the plant.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1—Schematic of interstock combinations with finger lime cultivars DPI 50-36 and Minnie in combination with two rootstocks (Volkamer and US-942) and Valencia scion IS=interstock.

FIGS. 2A-C. Photographs of A) Australian finger lime/Volkamer rootstock, B) Minnie finger lime/US-942 rootstock, C) Valencia scion/Minnie finger lime interstock/Volkamer rootstock.

### DESCRIPTION OF SPECIFIC EMBODIMENTS OF THE INVENTION

The present invention, in some embodiments thereof, relates to compositions and methods for mitigating HLB.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Huanglongbing (HLB) damages all parts of an orange tree. The disease may affect one or more of the leaf, twig, root system and fruit of the orange tree. Adverse effects of orange trees infected with the disease can progress from one branch of the tree to the entire tree. Infection often eventually affects plant viability, with infected plants typically surviving no more than about 3 to about 5 years. In some cases, infection of the orange tree with citrus greening eventually results in death of the trec.

Roots are often the earliest affected organs, such that by the time that above-ground organs exhibit symptoms, the disease has already progressed in the roots such that the plant is unlikely to survive for more than 3-5 years. While in the roots, the pathogen that causes citrus greening disease can replicate and damage the roots. Damaged roots can starve the trees, for example preventing or limiting absorption of nutrients from the soil. The pathogen can spread to other parts of the tree, spreading the damage to the other parts of the tree. In some instances, damage to roots of trees results in significant loss of the fibrous roots of the trees. Root systems of infected trees may be poorly developed. The disease may suppress new root growth. A tree may experience a loss of about 30% to about 50% of the fibrous root before symptoms are observed above-ground. In some cases, the bacterium colonizes the roots before spreading to the fruiting parts and/or leaf parts. Often symptoms in fruiting and/or leaf parts are manifested after root damage has already occurred. Although symptoms in fruiting parts may have the most direct economic impact, by the time symptoms are observed in leaf parts and/or fruiting parts of the tree, many trees have already suffered irreparable root damage. Restoration of the health of trees which have experienced irreparable root damage will often be difficult or unsuccessful, with the trees eventually dying. As such, direct fruit and/or leaf treatment may be insufficient to restore productivity of trees showing symptoms in its leaf parts and/or fruiting parts. Thus, a remedy that protects orange tree roots from damage shows greatest promise as a protection against HLB.

To overcome the deleterious effects of HLB and to provide an urgently needed immediate solution to citrus growers, the present inventors suggest using finger lime as an interstock to graft on to rootstocks known to be compatible with this and related species.

Without being bound by theory, it is suggested that the interstock-released metabolites will be transferred to the shoot and/or root system, thereby inducing or enhancing tree resistance\tolerance to HLB. Additionally, or alternatively the interstock serves as a barrier (due to its internal metabolite characteristics) or “filter”, inhibiting the pathogen and/or preventing the movement and distribution of the bacteria from the shoot into the root system. These mechanisms will also contribute to tree health as they will prevent/reduce infection of the roots which serve as reservoir for the pathogen and facilitate the chronic infection of the shoot system.

The present invention provides an affordable, environmentally friendly, and long-term solution to mitigate the devastating effects of HLB on citrus production by using novel grafting combinations of superior HLB tolerant/tolerant rootstocks and interstock to impart HLB tolerance to the scion Thus, according to a first aspect of the present invention there is provided a method of producing an HLB tolerant or resistant citrus rootstock, the method comprising grafting an HLB-tolerant or resistant citrus interstock on an HLB-susceptible or tolerant citrus rootstock grafted so as to obtain an HLB-susceptible or tolerant citrus rootstock grafted with the HLB-tolerant or resistant citrus interstock of a predetermined size, wherein the HLB-tolerant citrus interstock is of a microcitrus or a hybrid thereof.

The term “Citrus” or “citrus”, as used herein, refers to any plant of the genus Citrus, family Rutaceae, and includes Citrus volkameriana, Citrus maxima (Pomelo), Citrus medica (Citron), Citrus micrantha (Papeda), Citrus reticulata (Mandarin orange), Poncirus trifoliata (trifoliate orange), Citrus japonica (kumquat), Citrus australasica (Australian Finger Lime), Citrus australis (Australian Round lime), Citrus glauca (Australian Desert Lime), Citrus garrawayae (Mount White Lime), Citrus gracilis (Kakadu Lime or Humpty Doo Lime), Citrus inodora (Russel River Lime), Citrus warburgiana (New Guinea Wild Lime), Citrus wintersii (Brown River Finger Lime), Citrus halimii (limau kadangsa, limau kedut kera); Citrus indica (Indian wild orange), Citrus macroptera, and Citrus latipes. Hybrids also are included in this definition, for example Citrus x aurantiifolia (Key lime), Citrus x aurantium (Bitter orange), Citrus x latifolia (Persian lime), Citrus. limon (Lemon), Citrus x limonia (Rangpur), Citrus x paradisi (Grapefruit), Citrus x sinensis (Sweet orange), Citrus x tangerina (Tangerine), Poncirus trifoliata x C. sinensis (Carrizo citrange), and any other known species or hybrid of genus Citrus. Citrus known by their common names include, Imperial lemon, tangelo, orangelo, tangor, kinnow, kiyomi, Minncola tangelo, oroblanco, sweet orange, ugli, Buddha's hand, citron, lemon, orange, bergamot orange, bitter orange, blood orange, calamondin, clementine, grapefruit, Meyer lemon, Rangpur, tangerine, and yuzu, and these also are included in the definition of citrus or Citrus.

The term “Huanglongbing,” “Huanglongbing disease,” or “HLB,” (also referred to as citrus greening) as used herein, refers to a vector-transmitted disease of plants caused by microorganisms of the Candidatus genus Liberibacter, such as L. asiaticus, L. africanus, and L. americanus. This disease, for example, can be found in citrus plants, or other plants in the family Rutaceac. Symptoms of Huanglongbing disease include chlorosis and mottling of the leaves, occasionally with thickening of the leaves and reduced leaf size, canopy die-back, reduced fruit size, greening of the fruit, premature fruit drop, low fruit soluble solids content and poor flavor, and at advanced stages of the disease death of the plant. The bacterium Candidatus Liberibacter asiaticus, for example transmitted by psyllids, including Asian citrus psyllids and African citrus psyllids, is a likely cause of the disease.

Grafting is a technique which comprises joining of tissues from two or more different plants to form one composite plant. For example, grafting can include joining of plant segments from two different plants to form a graft union at the site where the two plant segments are joined. A vascular connection between the two plant segments is formed at the site of the graft union such that transport of water and/or nutrients occurs between the joined plant segments to enable growth of the composite plant. An upper part of a first plant, for example a scion, can be grafted onto a lower part of a second plant, for example a rootstock, to form the composite plant. Once grafting is complete, water and/or nutrients can be transported between the rootstock and the scion, such as to support growth of the scion.

Exemplary methods of grafting include, but are not limited to, whip-and-tongue grafting, double-tongue grafting, simple English grafting, complicated or double English grafting, splice grafting, T-grafting, V-grafting, W-grafting, side grafting, side-veneer grafting, cleft grafting, saddle grafting, bark grafting (vencer grafting), omega-grafting, chip-budding, occulation, stub grafting, shield budding, T-budding, inverted T-budding, patch budding, double shield budding, and flute budding.

Fusion of the graft can be confirmed by morphologically observing these tissues, particularly in a state where the parenchyma of both plants is fused (adhered), using a microscope or the like. Morphological observation can be performed, for example, by resin section observation of a section including the graft surface.

According to a particular embodiment, the grafting comprises inverted T-budding.

Typically, the rootstock is at least 10 cm in height at the time of grafting, at least 15 cm in height, at least 20 cm in height, at least 25 cm in height, at least 30 cm in height, at least 40 cm in height or even at least 50 cm in height.

Preferably, the join is made on the rootstock at a height of 15-20 cm from the soil.

The term “joint” as used herein refers to the connection formed between the grafted tissues, such as between a rootstock and an interstock; and an intertock and a scion.

The present inventors conceive that the grafting of the interstock to the rootstock according to embodiments of this aspect of the invention generates a rootstock that allows for the growth of a plant which will have a reduced bacterial titer or show less disease symptoms relative to root controls which have not undergone engraftment with the interstock.

As used herein, a “rootstock” refers to underground plant parts such as roots, from which new above-ground growth of a plant or tree can be produced. In accordance with the invention, a rootstock may be used to grow a different variety through asexual propagation or reproduction i.e., as grafting. The rootstock may be either HLB-tolerant or HLB-susceptible.

A number of sources for the HLB-tolerant rootstock are consistent with the disclosure herein. The HLB-tolerant rootstock can be derived from a feral citrus greening tolerant orange tree. The HLB-tolerant rootstock can be derived from a cultivated HLB-tolerant orange tree. The HLB-tolerant rootstock can be derived from a non-transgenic HLB-tolerant orange tree. The HLB-tolerant rootstock can be derived from a transgenic HLB-tolerant orange tree. The HLB-tolerant rootstock can be a rootstock derived from a seed of a feral or wild cultivar, and may be deposited in a public repository. The HLB-tolerant rootstock can be a rootstock derived from a plant sharing an allele present in a seed of a feral or wild cultivar, and may be deposited in a public repository and where the allele is absent from the interstock grafted to the rootstock or the fruiting cultivar scion grafted to the interstock. The HLB-tolerant rootstock can be a rootstock derived from a plant sharing an allele present in a seed of a feral or wild cultivar, where the allele is absent from the fruiting cultivar scion (and from the interstock), and where the rootstock is transgenic.

The HLB-tolerant rootstock can be a rootstock derived from a seed of a feral or wild cultivar. An exemplary seed stock is that deposited under ATCC Accession No. PTA-124301, although additional seed stocks are consistent with the present disclosure. The HLB-tolerant rootstock can be a rootstock derived from a US-942 (Citrus reticulata x Poncirus trifoliate plant.

A number of sources for the HLB-susceptible rootstock are consistent with the disclosure herein. These include rootstock derived from the following cultivars:

Volkamer, Washington Navel, Valencia, Trovita, Hamlin, Shamouti, Parson Brown, Pineapple, Queen, Sunstar, Gardner, and Mid-Sweet, Blood Oranges, Lue Gim Gong, Rhode Island Valencia and/or the Homosassa.

As used herein, an “interstock” refers to a heterologous plant part inserted between scion and stock in grafting, regardless of its size and length and age. The interstock is from a different plant variety than that of the scion and rootstock and is tolerant or resistant to HLB.

The interstock may be of any length, for example at least 5 mm, at least 1 cm, at least 2 cm or longer.

Examples of citrus which are tolerant or resistant to HLB from which the interstock may be derived are provided herein above.

According to a particular embodiment, the interstock is derived from a microcitrus.

Examples of Microcitrus are provided herein below:
Australian finger lime (Microcitrus australasica)(CRC 3661)
Australian finger lime (Microcitrus australasica)(CRC 3664)
Australian finger lime (Microcitrus australasica)(CRC 3670)
Australian finger lime (Microcitrus australasica)(CRC 3671)
Australian finger lime (Microcitrus australasica)(CRC 4108)
Australian red pulp finger lime (Microcitrus australasica)(CRC 1484)
Australian red pulp finger lime (Microcitrus australasica)(CRC 3672)
Australian round lime (Microcitrus australis)(CRC 3663)
Australian round lime (Microcitrus australis)(CRC 3665)
Australian round lime (Microcitrus australis)(CRC 3666)
Australian round lime (Microcitrus australis)(CRC 3668)
Australian round lime (Microcitrus australis)(CRC 3669)
Australian round lime (Microcitrus australis)(CRC 3673)
Australian round lime (Microcitrus australis)(CRC 4106)
Eremocitrus glauca (Australian desert lime)
Faustrimedin (xMicrocitronella sp.)
Microcitrus garrawayae (Garroway's Australian wild lime)(RSD 2002021)
Microcitrus garrawayae (Garroway's Australian wild lime)(RSD 2002022)
Microcitrus garrawayae (Garroway's Australian wild lime)(RSD 2002025)
Microcitrus inodora (large leaf Australian wild lime)(CRC 3784)
Microcitrus inodora (large leaf Australian wild lime)(CRC 3785)
Microcitrus papuana (Brown river finger lime)
Microcitrus warburgiana (New Guinea wild lime)(CRC 3298)
Microcitrus warburgiana (New Guinea wild lime)(CRC 3782)
Microcitrus virgata (Sydney hybrid)

In another embodiment, the microcitrus is a finger lime (e.g. Australian Finger Lime (Citrus australasica) or hybrids thereof (e.g. Minnie finger lime (Microcitrus australasica x M. inodora).

A number of finger limes are registered with the Australian Cultivar Registration Authority [wwwdotanbgdotgovdotau/area]: ‘Alstonville’, ‘Blunobia Pink Crystal’, ‘D1’ ‘Durham's Emerald’, ‘Judy's Ever-bearing’, ‘Rainforest Pearl’, ‘Pink Ice’, ‘Byron Sunrise’, and ‘Jali Red’. As used herein, a “scion” refers to a plant part that is grafted onto a rootstock variety (either directly or indirectly via an interposing interstock). A scion may be from the same as or a different plant type or variety than the rootstock.

As used herein “HLB-tolerant” refers to an infected, non-symptomatic, which allow a profitable commercial sustainable orchard.

As used herein “HLB-resistant” refers to HLB bacterium-free (i.e., non-infected, with non-detectable levels of HLB) plants which allow a profitable commercial sustainable orchard.

“Non-symptomatic refers to the lack of clearly definable disease symptoms, which may or may not be associated with reduced pathogen concentrations.

As used herein “HLB-susceptible” refers to the inability to allow profitable sustainable commercial citrus production.

“Symptomatic refers to the expression of disease symptoms following infection with the pathogen and usually associated with high concentrations of the pathogen.

Following generation of a composition comprising an HLB-susceptible or tolerant citrus rootstock grafted with an HLB-tolerant or resistant citrus interstock, the hybrid is left to grow under suitable conditions (e.g. at least one month, at least two months, at least three months or more) to produce a strong and sturdy joint between the two.

Once the joint is established, the method may proceed with grafting an HLB-susceptible scion thereto.

Typically, the second grafting step is carried out when the interstock is of a particular height (e.g. at least 0.5 cm, at least 1 cm, at 2 cm, at least 3 cm, at least 4 cm, at least 5 cm) and/or having a particular stem width (e.g. at least 2 mm).

The HLB susceptible scion may be derived from any citrus plant known to be susceptible to HLB (including those described herein above).

According to a particular embodiment, the HLB susceptible scion is derived from a sweet orange producing cultivar such as Washington Navel, Valencia, Trovita, Hamlin, Shamouti, Parson Brown, Pineapple, Queen, Sunstar, Gardner, and Mid-Sweet, Blood Oranges, Lue Gim Gong, Rhode Island Valencia and/or the Homosassa.

According to a particular embodiment, the HLB susceptible scion is derived from Valencia sweet orange (C. sinensis).

By carrying out the methods of the present invention, a composite citrus tree may be generated comprising a fruiting cultivar scion grafted onto a HLB-tolerant or resistant citrus interstock, which in turn is grafted onto an HLB-susceptible or tolerant citrus rootstock.

The resulting composite orange tree can demonstrate desired resistance to HLB. The composite fruit tree can be free or substantially free of one or more symptoms of HLB as described herein after being exposed to vectors of pathogens and/or pathogens which cause HLB. The composite fruit tree may be free or substantially free of one or more symptoms of the HLB without or substantially without applying to the composite fruit tree treatment for the disease, including for example pesticide which treats or mitigates the disease. The pesticide may include one or more of insecticides (e.g., for impairing and/or killing psyllids which transmit the bacterium Candidatus Liberibacter asiaticus), bactericides (e.g., for impairing and/or killing the bacterium Candidatus Liberibacter asiaticus) and larvicides (e.g., for impairing and/or killing larvae of psyllids). The composite fruit tree may demonstrate desired resistance without having been contacted with a dosage regimen of pesticide sufficient to prevent, alleviate, and/or eliminate the HLB.

The composite fruit tree may be free or substantially free of all symptoms of HLB without or substantially without being treated for the disease. For example, the composite orange tree may be able to maintain desired productivity of sweet oranges, such that the composite orange tree is a commercially viable sweet orange producing tree. The composite orange tree can be resistant to

HLB such that it can produce a desired quantity of fruits which ripen into sweet oranges. The desired productivity and/or sweetness of fruits suited for commercial viability will be understood by a person of ordinary skill in the art.

The composite fruit tree may be free or substantially free of other symptoms of the HLB after exposure to pathogens which cause the disease, including asymmetric chlorosis, abnormal coloration of the peel, curvature of the central core, and/or bearing irregularly shaped fruits. For example, the peel of the ripened oranges produced by the composite orange tree may be entirely orange in color and/or be free or substantially free of yellow coloring at the peduncle (stem) of the fruit. The central core of the ripened fruits may be free or substantially free of any curvature.

The composite orange tree may be 100%, 95%, 90%, 85%, 80%, 75%, 70%, or 60% productive and able to maintain desired productivity of sweet oranges. Productivity of the composite orange tree may be compared to a composite orange tree not exposed to HLB. Productivity of the composite orange tree may be compared to an orange tree treated with a pesticide so as to be protected from HLB. Productivity of the composite orange tree may be compared to a cultivated orange tree not exposed to HLB. In some cases, productivity of the composite tree is compared to a commercial orange tree that is non-resistant exposed or not exposed to HLB.

In some cases, the composite fruit tree comprises a scion that shows some symptoms of HLB. The scion can remain commercially viable despite showing symptoms of HLB. For example, the composite fruit tree maintains desired or commercially relevant production of sweet fruit. In some embodiments, the composite fruit tree can maintain desired productivity of sweet fruit about a year to about 5 years or more than 5 years after exposure to HLB, including about 2 years, about 3 years or about 4 years, about 5 years or more than about 5 years. The composite trec may remain commercially viable 1 year to about 5 years or more than about 5 years after exposure to the HLB, including about 2 years, about 3 years or about 4 years, about 5 years or more than about 5 years. In some embodiments, the composite fruit tree can produce commercially viable fruits without or substantially without use of pesticides which target HLB, or with use of a reduced quantity of such pesticides. In some embodiments, the composite fruit tree can produce commercially viable fruits with use of up to about 95%, about 90%, about 85%, about 80%, about 70%, or about 60% of the quantity of pesticides sufficient to treat commercial citrus trees such that the commercial citrus trees can maintain commercial viability, such as a standard quantity of pesticides. For example, a composite citrus tree which has not been treated with any pesticides or with a reduced quantity of pesticides can maintain productivity of sweet fruit after at least about 1 year, about 2 years, about 3 years, about 4 years, or about 5 years, after exposure to HLB, such that the composite tree is considered commercially viable. In some embodiments, the composite tree may exhibit some symptoms of HLB while maintaining commercial viability after at least about 1 year, about 2 years, about 3 years, about 4 years, or about 5 years, after exposure to HLB, without being treated with pesticides or with treatment of reduced quantities of pesticides.

A method of growing a composite citrus tree as described herein can include grafting a fruiting cultivar scion which provides the desired fruit (e.g. sweet orange) to an HLB tolerant or resistant interstock, which has in turn been grafted to HLB-susceptible or tolerant citrus rootstock. Grafting of the can be performed according to one or more processes known to one skilled in the art. The fruiting cultivar scion can be selected based on various characteristics of the fruiting cultivar, including a desired productivity of the cultivar and/or sweetness of fruits produced by the cultivar. The fruiting cultivar scion may be a high-yield cultivar. For example, the fruiting cultivar scion may be one or more commercially available cultivars as described herein.

Growing of the trees may be carried out in areas known to be affected by HLB. Preferably, the trees are grown under conditions which promote the growth of fruit-i.e. appropriate watering conditons, sun exposure, temperature, soil composition, soil moisture, wind, humidity, and soil pH. In one embodiment, the trees are grown in the absence of pesticides known to be effective against HLB. In another embodiment, the trees are grown in the presence of pesticides known to be effective against HLB.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Rootstocks were selected based on their good performance in combination with Minnie finger lime (Bowman et al., 2019) and the popular US-942, which has been the most popular rootstock in Florida since the 2018/19 production season (Rosson, 2020). The popularity of US-942 is related to its documented HLB tolerance (Albrecht and Bowman 2012) and its superior field performance in all Florida citrus production regions (Bowman et al. 2015, 2016a, 2016b). Because of its high vigor, Volkamer is exceptionally suited as a rootstock for finger limes (in scion and interstock position). The compatibility of these two rootstocks with the Microcitrus genus was documented in Bowman et al. (2019) and is evident in current propagations and ongoing field studies. Valencia sweet orange (C. sinensis) is the scion that is used for more than 90% of citrus production in Florida (Rosson, 2020). Sweet oranges are among the most HLB-susceptible citrus species (McClean and Schwarz, 1970; Folimonova et al., 2009) in contrast to many mandarin x trifoliate orange hybrids like US-942 (Albrecht and Bowman, 2012; Albrecht et al., 2014; Boava et al., 2015).

Rootstock liners were grafted (budded) with Minnie finger lime (Microcitrus australasica x M. inodora) and DPI 50-36 (M. australasica). Both M. australasica and M. inodora were found to be highly tolerant to HLB based on six years of field performance (Dutt et al., 2017b; Ramadugu et al., 2016). Valencia was used as an interstock/grafting control. After the interstocks reached an adequate size, at least 5 mm in length, they were grafted with Valencia scion.

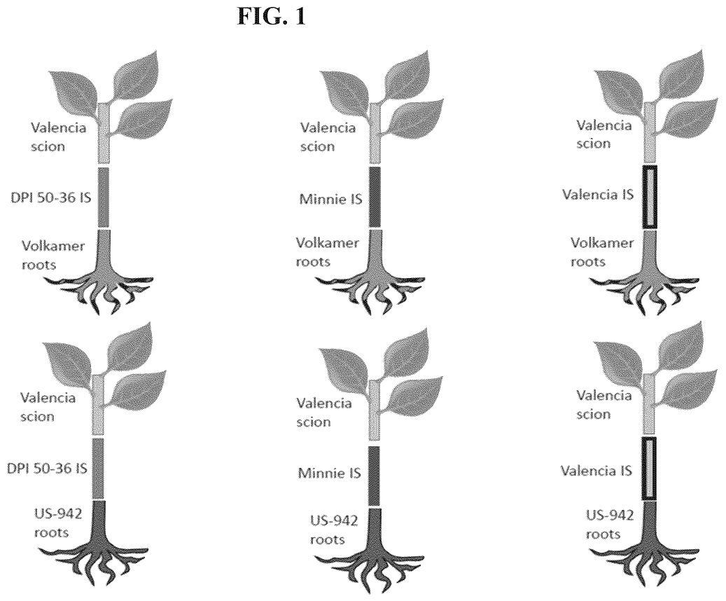

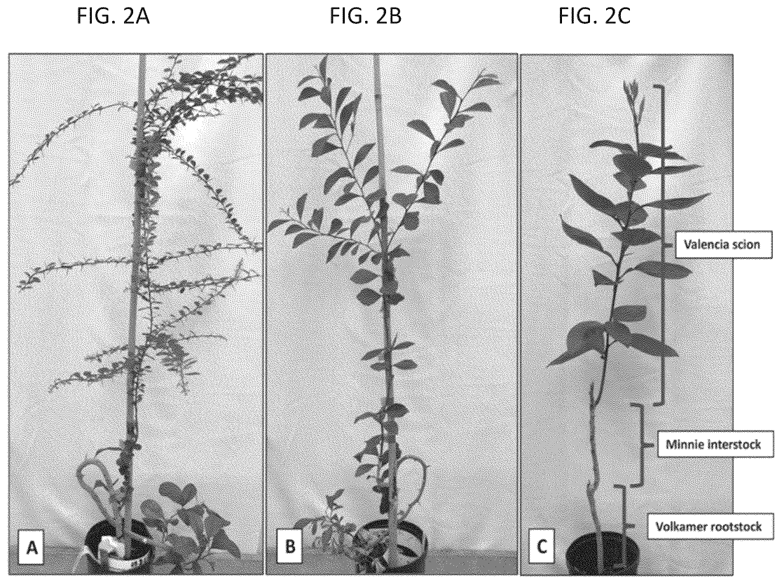

The different graft combinations are depicted in FIG. 1 and include the following six combinations: 1) Valencia/DPI-50-36/Volkamer, 2) Valencia/Minnic/Volkamer, 3) Valencia/Valencia/Volkamer, 4) Valencia/DPI-50-36/US-942, 5) Valencia/Minnie/US-942, and 6) Valencia/Valencia/US-942. FIG. 2 shows different stages of the grafting process.

Twenty plants of each graft combination were propagated; of these 6-8 plants with uniform growth will be selected for further experimentation upon start of the proposed project.

Metabolomic interactions of rootstocks and scions and grafting effects which include rootstock and HLB effects on tolerant and susceptible citrus species are further evaluated as well as tissue specific metabolomics and rootstock-scion reciprocal effects on metabolite transport and HLB progression.

Rootstocks (Volkamer lemon [Citrus volkameriana] and US-942 [C. reticulata x Poncirus trifoliata]) will be grown from seeds in 3.8 cm x 21 cm cone cells (Cone-tainers; Stuewe and Sons) containing a peat-based potting medium (Pro Mix BX; Premier Horticulture, Inc.). Rootstocks will be grown for 4-6 months until they are large enough to be grafted. Disease-free certified buds of Minnie finger lime (Microcitrus australasica x M. inodora; Bowman et al., 2019) and of Australian finger lime (Microcitrus australasica) FDACS/DPI clone 50-36 will be grafted using the inverted T-method (Albrecht et al., 2021). Control plants will be budded with a standard Valencia clone (1-14-19) to be used as a graft/interstock control. Buds will be inserted into the rootstock at a height of 15-20 cm from the soil. Once interstocks have reached the desirable length and stem diameter, they will be budded with Valencia Jan. 14, 2019 to be used as scion. The final interstock length will be 15 cm. Plants will be grown under natural light conditions in a temperature-controlled greenhouse, irrigated by hand as needed, and fertilized using a water-soluble fertilizer with micronutrients (20N-10P-20K) at a rate of 400 mg N/L. Insecticides will be applied as needed. Trees propagated in this manner will be transferred to a citrus orchard located in southwest Florida at the UF/IFAS Southwest Florida Research and Education Center to assess the general horticultural potential of these new types of trees. The SWFREC citrus orchard is situated in a location characterized by heavy psyllid pressure; therefore we will also be able to compare the HLB tolerance of trees containing a finger lime interstock with that of trees containing a Valencia interstock using the methods described below.

Field trials. Plants of all six rootstock/interstock/scion combinations will be produced using standard horticultural practices as described. Field-ready plants (approximately 12 months after start of propagations) will be transferred to the SWFREC citrus research orchard. Half of the plants will be retained in the healthy state to be used for clearly defining disease effects and comparing metabolomic profiles. To achieve this, all trees will be covered with individual protective covers (IPCs; Alferez et al.,2021) made of netting with a mesh size small enough to prevent psyllids from accessing the plants. After a brief adaptation period, all plants will be slightly pruned to generate new flush attractive to the psyllids. Plants that are to be infected with CLas will be uncovered for several weeks to attract psyllids and ensure infestation; trees to be used as healthy controls will remain covered. To prevent shading effects among treatments, IPCs will be put back on the infected plants, but holes will be cut into the mesh to allow psyllids to enter. Based on previous field experiments conducted by the Albrecht lab at this location and in this manner, infection of plants is expected to occur within the first months after planting. Plants will be monitored regularly for presence and density of the psyllid vector using established methods (Monzó et al., 2015). Foliar HLB symptoms will be assessed by visual rating as described in Kunwar et al. (2021). Leaf CLas titers will be determined in 3-month intervals using established methods (Albrecht and Bowman, 2019). Briefly, DNA will be extracted using the DNAcasy Plant Mini kit (Qiagen) and real-time PCR assays will be performed using primers HLBas and HLBr and probe HLBp (Li et al., 2006) and primers COXf and COXr and probe COXp for normalization and DNA control. PCR reactions will be conducted with an Applied Biosystems QuantStudio 3 Real-Time PCR system. Once infection has been confirmed in all plants, 3-4 mature and fully expanded leaves will be collected from each plant, immediately frozen in liquid nitrogen, and stored at-80° C. until metabolite extraction. Leaves will be collected 1) after the spring flush once they are fully expanded and 2) in fall/winter, when HLB foliar discase symptoms are most expressed in susceptible scions. Basic horticultural traits (tree height, canopy volume, trunk diameter) will be assessed biannually. Other physiological parameters (leaf nutrients, stomatal conductance, root density, etc.) will be evaluated seasonally as resources are available.

Greenhouse studies. These studies will be conducted in support of the field studies to decipher the reciprocal relationship of the grafting partners and consequences for HLB tolerance. Plants will be propagated as described above. During the propagation process, leaf tissue from the different grafting partners will be collected for metabolomics analysis as follows. During the grafting process and after the bud union is well connected to the rootstock or interstock, respectively (10-14 days), the stock with leaves attached is bent to remove apical dominance effects and promote bud growth (Albrecht et al., 2021). In general, the stock is cut off after several weeks when the scion has developed its own leaves to provide photosynthates for growth. For our study, however, we will leave the stocks with the leaves attached. Once the scion is well-developed, we will sample a minimum of three mature, fully expanded leaves from each grafting partner (scion, interstock, and rootstock) for metabolomics analysis. Following leaf sampling, rootstock and interstock are cut off at 5 cm near their respective graft unions, and bark tissuc containing the phloem and the outer (active) ring of the xylem will be excised from the stems to be used for metabolomics analysis and comparison with the leaves. All tissues will be frozen immediately in liquid nitrogen after excision and stored at −80° C. until processed. A minimum of six plants will be used for each graft combination.

Field studies. Plants of all six rootstock/interstock/scion combinations will be produced using standard horticultural practices as described above. Twelve trees of each rootstock/interstock/scion combination will be planted at the SWFREC research orchard, half of which will be maintained as healthy plants and half as infected plants as described aboc. Once infection has been confirmed by PCR (and control trees confirmed as non-infected) and foliar symptoms characteristic of HLB (blotchy mottle) begin to appear in the most susceptible trees (those with sweet orange interstock), leaves will be collected from each tree as described above and immediately frozen in liquid nitrogen and stored at −80° C. to be used for metabolomics.

Metabolomics analysis. Metabolomic analysis will be performed according to our published methods (Tietel et al., 2020). Samples will be stored at −80° C. and kept cold during the extraction process. Upon derivatization, samples will be analyzed in an Agilent 7890 gas chromatograph coupled to a mass spectrometer (GC-MS) instrument. Once the GC-MS data has been collected, data will be integrated and aligned, and compounds annotated based on the National Institute for Science and Technology (NIST) library, as well as an in-house library based on commercial standards. This will be done manually (with the aid of Agilent Unknown Analysis and MassHunter Quant software packages). The manual process will facilitate the verification and annotation of each compound, which is crucial in the search for biomarkers. Analysis will then be performed using MetaboAnalyst and JMP software, and based on large data informatics, e.g., PCA/PLSDA, biomarker analysis, fold change analysis, ANOVA/t-test, volcano plots, and heatmaps, as well as discriminant analysis.

For the current work, an untargeted approach will be adopted. Such an approach will enable comparison between the metabolomic profiles of the tolerant grafted combinations and susceptible ones, to identify unique metabolites, only appearing in the tolerant trees. Untargeted analysis might also result in including some unknown compounds in the dataset, which could later be annotated by using commercial standards. For the analysis, the experimental design will include at least six biological replicates of each treatment (combination) to account for natural diversity and allow a reliable statistical analysis.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited in the Document

Adhikari B., M. Dutt, T. Vashisth. 2021. Comparative phytochemical analysis of the fruits of four Florida-grown finger lime (Citrus australasica) selections. LWT, 135, p.110003.
Albrecht, U. and L. G. Archer. 2021. Principles and risks of trunk injection for delivery of crop protection materials. Citrus Industry Magazine 102(5): 14-17
Albrecht, U. and K. D. Bowman. 2019. Reciprocal influences of rootstock and scion citrus cultivars challenged with Ca. Liberibacter asiaticus. Sci. Hortic. 254:133-142.
Albrecht, U. and K. D. Bowman. 2012. Tolerance of trifoliate citrus rootstock hybrids to
Candidatus Liberibacter asiaticus. Sci. Hortic. 147:71-80.
Albrecht, U. and K. D. Bowman. 2011. Tolerance of the trifoliate citrus hybrid US-897 (Citrus reticulata x Poncirus trifoliata) to huanglongbing. HortScience 46:16-22
Albrecht U., M. Zekri, J. Williamson. 2021. Citrus propagation. www(dot)edis(dot)ifas(dot)ufl(dot)edu/publication/HS1309
Albrecht, U., D. G. Hall, K. D. Bowman. 2014. Transmission efficiency of Candidatus Liberibacter asiaticus and progression of huanglongbing disease in graft-and psyllid-inoculated citrus. HortScience 49(3): 367-377.
Alferez F., O. Batuman, S. G. Gaire, U. Albrecht, J. Qureshi. 2021. Assessing deployment patterns for individual protective covers (IPCs). Citrus Industry Magazine. 102(8): 14-16.
Alquézar B., H. X. L. Volpe, R. F. Magnani, M. P. de Miranda, M. A. Santos, N. A. Wulff, J/M. S. Bento, J. R. P. Parra, H. Bouwmeester, L. Peña. 2017. β-caryophyllene emitted from a transgenic Arabidopsis or chemical dispenser repels Diaphorina citri, vector of Candidatus Liberibacters. Sci. Rep 7(1): 1-9.
Baldwin, E., A. Plotto, J. Manthey, G. McCollum, J. Bai, M. Irey, R. Cameron, G. Luzio. 2010. Effect of Liberibacter infection (huanglongbing disease) of citrus on orange fruit physiology and fruit/fruit juice quality: chemical and physical analyses. J. Agric. Food Chem. 58(2): 1247-1262.
Boava, L. P., C. H. D. Sagawa, M. Cristofani-Yaly, M. A. Machado. 2015. Incidence of ‘CandidatusLiberibacter asiaticus’-infected plants among citrandarins as rootstock and scion under field conditions. Phytopathology 105:518-524.
Bové, J. M. 2006. Huanglongbing: a destructive, newly emerging, century-old disease of citrus. J. Plant Pathol. 88:7-37.
Bowman, K. D., G. Mc Collum, A. Plotto, J. Bai. 2019. Minnie finger lime: a new novelty citrus cultivar. HortScience 54(8): 1425-1428.
Bowman, K. D. and G. McCollum. 2015. Five new citrus rootstocks with improved tolerance to huanglongbing. HortScience 50:1731-1734.
Bowman, K. D., G. McCollum, U. Albrecht. 2016a. Performance of ‘Valencia’ orange (Citrus sinensis [L.] Osbeck) on 17 rootstocks in a trial severely affected by huanglongbing. Sci. Hortic. 201:355-361
Bowman, K. D., L. Faulkner, M. Kesinger. 2016b. New citrus rootstocks released by USDA 2001-2010: Field performance and nursery characteristics. HortScience 51(10): 1208-1214.
Da Graça, J.V. 1991. Citrus greening disease. Annu. Rev. Phytopathol. 19:109-136.
Dutt M. 2021. Could finger limes be Florida's hero?
www(dot)citrusindustry(dot)net/2021/03/05/could-finger-limes-be-floridas-hero/Dutt, M., E. Nielsen, Q. Yu, F. Gmitter, J. W. Grosser. 2017a. Utilizing Australian lime genetics for citrus improvement. 2017 ASHS Annual Conference.
Dutt M., E. Nielsen, J. Grosser. 2017b. Finger lime could be new crop for citrus growers. Citrus Industry magazine 98(3): 22-25. www(dot)ashs(dot)confex(dot)com/ashs/2017/webprogramarchives/Paper27348(dot)html
FAO. Food and Agricultural Organization of the United Nations. 2020; Available from: www(dot)faostat3(dot)fao(dot)org/faostat −gateway/go/to/download/Q/QC/E.
Folimonova, S. Y., C. J. Robertson, S. M. Garnsey, S. Gowda, W. O. Dawson. 2009. Examination of the responses of different genotypes of citrus to huanglongbing (citrus greening) under different conditions. Phytopathology 99(12): 1346-1354.
Garnier, M., N. Danel, J. M. Bové. 1984. The greening organism is a gram-negative bacterium. IOCV Conference Proceedings 9(9): 115-124.
Gottwald, T. R., J. V. D. Graça, R. B. Bassanezi. 2007. Citrus huanglongbing: the pathogen and its impact. Plant Health Prog. 8(1): 31.
Graham, J., T. Gottwald, T., M. Sctamou. 2020. Status of huanglongbing (HLB) outbreaks in Florida, California and Texas. Trop. Plant Pathol. 45:265-278.
Hall, D. G., M. L. Richardson, E. D. Ammar, S. E. Halbert. 2013. Asian citrus psyllid, Diaphorina citri, vector of citrus huanglongbing disease. Entomol. Exp. Appl. 146(2): 207-223.
Hernández-Suárez, E., L. Suárez-Méndez, M. Parrilla, J. M. Arjona-López, A. Hervalejo, F. J.
Arenas-Arenas. 2021. Feeding and oviposition behaviour of Trioza erytreae (Hemiptera: Triozidac) on different citrus rootstock material available in Europe. Insects 12(7): 623.
Hu J., J. Jiang, N. Wang. 2018. Control of citrus huanglongbing via trunk injection of plant defense activators and antibiotics. Phytopathology 108:186-195.
Huang, C.-Y., K. Araujo, J. Niño Sanchez, G. Kund, J. Trumble, C. Roper, K. E. Godfrey, H. Jin. 2021. A stable antimicrobial peptide with dual functions of treating and preventing citrus huanglongbing. PNAS 118(6): c2019628118.
Israel Farmer Federation. 2015. Data on the Israeli agriculture industry. www(dot)mhh(dot)org(dot)il/uploads/n/file 1067(dot)pdf.
Killiny, N., S. E. Jones, Y. Nchela, F. Hijaz, M. Dutt, F. G. Gmitter, J. W. Grosser. 2018. All roads lead to Rome: Towards understanding different avenues of tolerance to huanglongbing in citrus cultivars. Plant Physiol. Biochem. 129:1-10.
Killiny, N., F. Hijaz, P. Gonzalez-Blanco, S. E. Jones, M. O. Pierre, C. Vincent. 2020. Effect of adjuvants on oxytetracycline uptake upon foliar application in citrus. Antibiotic 9(10): 677.
Kunwar, S., J. Grosser, F. G. Gmitter, W. S. Castle, U. Albrecht. 2021. Field performance of ‘Hamlin’ orange trees grown on various rootstocks in huanglongbing-endemic conditions. HortScience, 56(2): 244-253.
Li J., V. G. Kolbasov, Z. Pang, S. Duan, D. Lec, Y. Huang, J. Xu, D. Teper, T. Lamichhane, N. Wang. 2021. Evaluation of the control effect of SAR inducers against citrus Huanglongbing applied by foliar spray, soil drench or trunk injection. Phytopathol. Res. 3:2.
Liao H.-Y. and J. K. Burns. 2012. Gene expression in Citrus sinensis fruit tissues harvested from huanglongbing-infected trees: comparison with girdled fruit. J. Exp. Bot. 63(8): 3307-3319.
Luo, X., A. L. Yen, K. S. Powell, F. Wu, Y. Wang, L. Zeng, Y. Yang, Y. Cen. 2015. Feeding behavior of Diaphorina citri (Hemiptera: Liviidae) and its acquisition of ‘Candidatus Liberibacter asiaticus’, on huanglongbing-infected Citrus reticulata leaves of several maturity stages. Florida Entomologist, pp. 186-192.
McClean, R. E. and A. P. D Schwarz (1970). Greening or blotchy-mottle disease of citrus. Phytophylactica, 2(3): 177-194.
Merfa, M. V., E. Perez-López, E. Naranjo, M. Jain, D. W. Gabriel, L. de La Fuente. 2019. Progress and obstacles in culturing ‘Candidatus Liberibacter asiaticus’, the bacterium associated with huanglongbing. Phytopathology 109:1092-1101.
Monzó C., H. A. Arevalo, M. M. Jones, P. Vanaclocha, S. D. Croxton, J. A. Qureshi, P. A. Stansly. 2015. Sampling methods for detection and monitoring of the Asian citrus psyllid (Hemiptera: Psyllidac). Environ. Entomol. 44(3): 780-788.
Ramadugu, C., M. L. Keremane, S. E. Halbert, Y. P. Duan, M. L. Roose, E. Stover, R. F. Lec. 2016. Long-term field evaluation reveals huanglongbing resistance in citrus relatives. Plant Disease 100(9): 1858-1869.
Robinson, A. 2021. Finger Limes and HLB? In UCANR: Promoting economic prosperity in California. www(dot)ucanr(dot)edu/blogs/blogcore/postdetail(dot)cfm?postnum=46159.
Rosson, B. 2020. Florida Department of Agriculture and Consumer Services. Citrus budwood annual report 2019-20. https://www(dot)fdacs(dot)gov/content/download/94009/file/2019-2020-Annual-Report(dot)pdf.
Singerman, A., M. Burani-Arouca, S. H. Futch. 2018. The profitability of new citrus plantings in Florida in the era of huanglongbing. HortScience 53(11): 1655-1663.
Shi, Q., J. George, J. Krystel, S. Zhang, S. L. Lapointe, L. L. Stelinski, E. Stover, E. 2019. Hexaacetyl-chitohexaose, a chitin-derived oligosaccharide, transiently activates citrus defenses and alters the feeding behavior of Asian citrus psyllid. Horti. Res.6(1): 1-10.
Shokrollah, H., T. L. Abdullah, K. Sijam, K., S. NA. Abdullah, N. A. P Abdullah. 2009. Differential reaction of citrus species in Malaysia to huanglongbing (HLB) disease using grafting method. Amer. J. Agri. Biol. Sci. 4(1): 32-38.
Stansly, P. A., H. A. Arevalo, J. A. Qureshi, M. M. Jones, K. Hendricks, P. D. Roberts, F. M. Roka. 2014. Vector control and foliar nutrition to maintain economic sustainability of bearing citrus in Florida groves affected by huanglongbing. Pest Manag. Sci. 70(3): 415-426.
Tietel, Z., Srivastava, S., Fait, A., Tel-Zur, N., Carmi, N., Raveh, E., 2020. Impact of scion/rootstock reciprocal effects on metabolomics of fruit juice and phloem sap in grafted Citrus reticulata. PloS one, 15(1): p.e0227192.
Wang, N. (2019). The citrus huanglongbing crisis and potential solutions. Mol. Plant 12(5): 607-609.
Weber, K. C., W. Qiu, J. W. Grosser, M. Dutt. 2019. Comparative transcriptome analyses between the HLB tolerant Australian finger lime and HLB susceptible Valencia sweet orange. 2019 ASHS Annual Conference.

1. A composition comprising an HLB-susceptible or tolerant citrus rootstock grafted with an HLB-tolerant or resistant citrus interstock.

2. The composition of claim 1, comprising an HLB-susceptible or tolerant citrus rootstock grafted with an HLB-tolerant or resistant citrus interstock and optionally an HLB-susceptible scion, wherein said HLB-tolerant citrus interstock is of a microcitrus or a hybrid thereof.

3. A method of producing an HLB tolerant citrus, the method comprising:
(a) grafting an HLB-tolerant or resistant citrus interstock on an HLB-susceptible or tolerant citrus rootstock grafted so as to obtain an HLB-susceptible or tolerant citrus rootstock grafted with said HLB-tolerant or resistant citrus interstock of a predetermined size, wherein said HLB-tolerant citrus interstock is of a microcitrus or a hybrid thereof; and
(b) grafting an HLB-susceptible scion on said HLB-susceptible or tolerant citrus rootstock grafted with said HLB-tolerant or resistant citrus interstock.

4. A method of producing an HLB tolerant or resistant citrus rootstock, the method comprising grafting an HLB-tolerant or resistant citrus interstock on an HLB-susceptible or tolerant citrus rootstock grafted so as to obtain an HLB-susceptible or tolerant citrus rootstock grafted with said HLB-tolerant or resistant citrus interstock of a predetermined size, wherein said HLB-tolerant citrus interstock is of a microcitrus or a hybrid thereof.

5. The composition of claim 1, one wherein said citrus is any cultivar from the genus Citrus, including but not limited to sweet orange (C. sinensis), lemon (C. limon), lime (C. latifolia) grapefruit (C. paradise), sour orange (C. aurantium), and mandarin (C. reticulata), trifoliate orange (Poncirus trifoliata) and any other citrus varieties and their hybrids.

6. The method of claim 3, wherein said interstock of said predetermined size is above 0.5 cm.

7. The method of claim 3, wherein said interstock of said predetermined size is above 0.6 cm.

8. The composition of claim 1, wherein said HLB-tolerant citrus interstock is of a finger lime or hybrids thereof.

9. The composition of claim 8, wherein said finger lime is Minnie finger lime or hybrids thereof.

10. The composition of claim 2, wherein said scion is of Valencia sweet orange (C. sinensis).

11. The composition of claim 1, wherein said rootstock is of Volkamer lemon [Citrus volkameriana] or US-942 (Citrus reticulata x Poncirus trifoliata).

12. The composition of claim 1, wherein said rootstock is of US-942 (Citrus reticulata x Poncirus trifoliata).

13. The composition of claim 1, wherein at least one of said rootstock, scion and interstock is diploid.

14. The composition of claim 2, being a tree.

15. A The_composition of claim 1, wherein said an HLB-susceptible or tolerant citrus rootstock is grafted with a microcitrus which is HLB tolerant or resistant.

16. The composition of claim 15, wherein said microcitrus is Minnie finger lime or hybrids thereof.

17. A method of producing fruits, the method comprising:
(a) growing a plant from the composition of claim 2; and
(b) harvesting fruits from the plant.
